(19) United States
(12) Patent Application Publication
(10) Pub. No.: US 2024/0342429 A1
(43) Pub. Date: Oct. 17, 2024

(54) ADAPTATION OF SENSES DATASTREAMS IN VIRTUAL REALITY AND AUGMENTED REALITY ENVIRONMENTS

(71) Applicant: Telefonaktiebolaget LM Ericsson (publ), Stockholm (SE)

(72) Inventors: Athanasios KARAPANTELAKIS, SOLNA (SE); Divya SACHDEVA, NEW DELHI (IN); Lackis ELEFTHERIADIS, Valbo (SE); Maxim TESLENKO, SOLLENTUNA (SE); Konstantinos VANDIKAS, Solna (SE); Alexandros NIKOU, Danderyd (SE); Alessandro PREVITI, STOCKHOLM (SE); Kristijonas CYRAS, San Jose, CA (US)

(73) Assignee: Telefonaktiebolaget LM Ericsson (publ), Stockholm (SE)

(21) Appl. No.: 18/294,420

(22) PCT Filed: Aug. 4, 2021
(86) PCT No.: PCT/EP2021/071735

§ 371 Date: Feb. 1, 2024

(51) Int. Cl. A61M 21/02; A61M 21/00; G06F 3/01

(52) U.S. Cl. CPC A61M 21/02; G06F 3/011; A61M 2021/0016; A61M 2021/0022; A61M 2021/0027; A61M 2021/0044

(57) ABSTRACT

In one aspect, a computer-implemented method of adapting a sensory datastream is provided. The method includes obtaining a raw sensory datastream from a source, wherein the raw sensory datastream comprises input for a sensory actuator of a user device. The method includes obtaining state information, wherein the state information comprises information indicating a first state of a user. The method includes predicting, using a machine learning model, a desired second state of the user based on the obtained state information. The method includes determining an action to adapt the raw sensory datastream based on the desired second state of the user. The method includes adapting the raw sensory datastream in accordance with the determined action and the first state of the user to create a processed sensory datastream. The method includes providing the processed sensory datastream to the sensory actuator of the user device.

### TECHNICAL FIELD

Disclosed are embodiments related to adapting senses datastreams in augmented reality and virtual reality environments. Certain embodiments relate the internet of senses, machine learning, and reinforcement learning.

### INTRODUCTION

The internet of senses promises experiences utilizing all senses in virtual or augmented reality (VR/AR) environments, for example as described in Reference [1]. In such environments, users can interact with digital objects using one or more of their senses. From a network perspective, sensory input to a user's VR/AR device are sets of synchronized datastreams (i.e., sound, vision, smell, taste, and touch). In a VR/AR environment the end-user device feeds the data to the sensory actuators for human users to experience.

Considering sensory perception however, people perceive things differently. For example, hyperesthesia is a condition that involves an abnormal increase in sensitivity to stimuli of a particular sense (e.g., touch or sound). Hyperesthesia may have unwanted side-effects, for example some people may hear painfully loud noises where in reality the volume is not as high. Another condition, called misophonia, triggers unreasonable physiological or psychological responses as reaction to certain sounds (for example food munching). Finally, hypoesthesia, being the reverse to hyperesthesia, is a condition where sense perception is reduced. For example, a certain smell or sound may not be as strong. In addition to medical conditions, there are also personal preferences on taste, smell, touch, sound and/or view that may trigger a positive or a negative response.

Further to the above, Reference [9] uses sensory data from users themselves and from users' environment to generate user experiences. Reference is targeted at removing noise from images captured from a camera and correlating haptic feedback with the image content.

### SUMMARY

When it comes to gauging emotional state of users, there exist several approaches, from invasive to non-invasive. A comprehensive review is discussed in Reference [2]. The following list is approximately ranked from least to most invasive given the current state of the art-potentially in the future more invasive approaches will become easier to attain or such implants may be considered as the norm.

Heart Rate Variability (HRV) measures the time between heartbeats (heartrate), which may require fewer sensors than previous approaches. For example, wearables such as Apple watch can measure HRV.

Skin temperature measurements (SKT) measure the temperature at the skin (e.g., a sweating person would have higher skin temperature) and relate it to human emotional state.

Respiration Rate Analysis (RRA) measures the respiration velocity and depth. It is possible to implement RR with non-contact measurement methods such as video cameras and/or thermal cameras.

Facial Expressions (FE), body posture (BP) and gesture analysis (GA) are non-invasive methods that have been becoming increasingly popular the last few years due to advances in computer vision and specifically visual object detection and the use of convolutional neural networks. In these methods, images of the face and body are used, and algorithms detect and analyze different types of expressions and postures and correlate them with emotional states.

Electrooculography (EOG) uses either electrodes placed above or below the eye and the left and to the right, or special video cameras (such as video oculography systems and camera infrared oculography), to measure eye movement that could indicate an emotional state.

Electroencephalography (EEG) uses a special device called an electroencephalogram to collect EEG signals. This device contains electrodes attached to human scalp using an adhesive material or using a headset. A subsequent analysis of frequency ranges generated from EEG signals may identify different emotional states. Cutting-edge EEG devices may even be portable.

Electrocardiography (ECG) uses fewer sensors (compared to EEG), positioned on the human body—instead of measuring brain waves this method measures the electrical activity of the heart.

Galvanic Skin Response (GSR) measures electrical parameters of the human skin and requires several sensors placed in different parts of the body.

Electromyogram (EMG) uses electrodes to measure neuromuscular abnormalities, which could be triggered as an emotional reaction.

In Reference [9], there is no monitoring of the emotional state of the consumer/user to alternate the intensity of an existing datastream. Instead, contextual information such as foot-related pressure readings, as well as physiological characteristics such as shoulder width, arm length, muscle tone, vein signature, style of movement, etc. is obtained in order to do access control, i.e., permit or block datastreams from reaching the user.

In Reference [10], a marker indicator is embedded in an image frame (and not the emotional state of the user) to remove image noise and render haptic feedback to the user. In addition, haptic feedback is generated and not adjusted.

Accordingly, there is a deficiency in the state of art when it comes to adapting sensory input media streams (also to be referred henceforth as datastreams) in response to the emotional state of the consumer. Every consumer of the datastreams has different sensitivities and personal preferences, however, and embodiments disclosed herein learn those sensitivities and preferences over time in an automated, unsupervised way (i.e., without user feedback).

According to some embodiments, a system and method for learning to enhance or subdue sense-related data contained in datastreams based on the reactions of the user is provided. The embodiments disclosed herein provide an intuitive way of adapting the datastreams, which can improve the overall user experience of AR/VR and mixed reality applications.

In one aspect, a computer-implemented method of processing a stream of sensory data is provided. The method includes obtaining an input stream of sensory data from a source, wherein the input stream of sensory data comprises input for a sensory actuator of a user device. The method includes obtaining state information, wherein the state information comprises information indicating a first state of a user. The method includes determining, using a machine learning model, a desired second state of the user based on the obtained state information. The method includes determining an action to process the input stream of sensory data based on the desired second state of the user. The method includes generating an output stream of sensory data by processing the input stream of sensory data in accordance with the determined action. The method includes rendering the output stream of sensory data to the sensory actuator of the user device.

In another aspect there is provided a device adapted to perform the method. In another aspect there is provided a computer program comprising instructions which when executed by processing circuitry of a device causes the device to perform the methods. In another aspect there is provided a carrier containing the computer program, where the carrier is one of an electronic signal, an optical signal, a radio signal, and a computer readable storage medium.

One of the advantages made possible by the embodiments disclosed herein is the adaptation of the emotional effect that is produced by a datastream to a desired effect as learned from experience by way of reinforcement learning. This enhances the user Quality of Experience (QoE). Another advantage is improved privacy and preserving privacy of the user since the learning/adaptation may be done on the User Equipment (UE) side. Another advantage is that the embodiments allow growth of Internet of Senses applications to an audience that may be hyper or hypo sensitive to certain or all senses.

### BRIEF DESCRIPTION OF THE DRAWINGS

The accompanying drawings, which are incorporated herein and form part of the specification, illustrate various embodiments.

FIG. 1 is a generalized block diagram, according to some embodiments.

FIG. 2 is a block diagram of components of a system, according to some embodiments.

FIG. 3. Illustrates a mapping of emotions to a set of measurable dimensions, according to some embodiments.

FIG. 4 is a flowchart illustrating a process according to some embodiments.

FIG. 5 is a flowchart illustrating a process according to some embodiments.

FIG. 6 is a block diagram of an apparatus according to an embodiment.

### DETAILED DESCRIPTION

Embodiments disclosed herein relate to systems and methods for learning to enhance or subdue sense-related data contained in datastreams based on the reactions of the user. The embodiments disclosed herein provide an intuitive way of adapting the data streams, which can improve the overall user experience of mixed reality applications.

One of the advantages made possible by the embodiments disclosed herein is the adaptation of the emotional effect that is produced by a datastream to a desired effect as learned from experience by way of reinforcement learning. This enhances the user Quality of Experience (QoE). Another advantage is improved privacy and preserving privacy of the user since the learning/adaptation may be done on the User Equipment (UE) side. Another advantage is that the embodiments allow growth of Internet of Senses applications to an audience that may be hyper or hypo sensitive to certain or all senses.

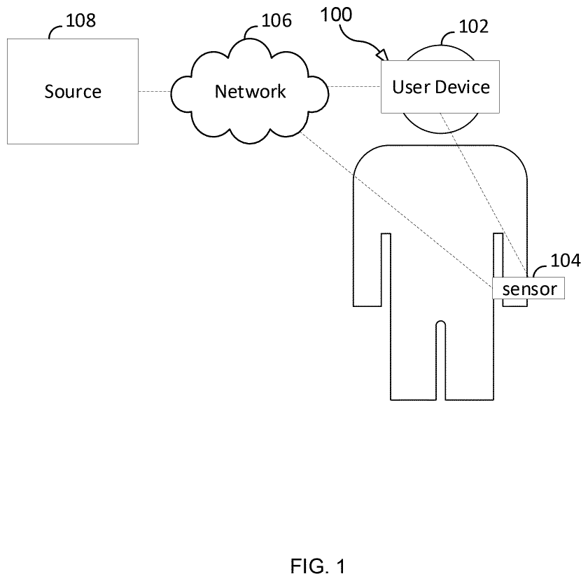

FIG. 1 is a generalized block diagram, according to some embodiments. As shown in FIG. 1, a user device 100 is in communication with a source 108 via network 106. In some embodiments, user device 100 is in communication with source 108 directly without network 106. The user device 100 may encompass, for example, a mobile device, computer, tablet, desktop, or other device used by an end-user capable of controlling a sensory actuator, such as a screen or other digital visual generation devices, digital scent generator capable of creating aroma or scent, taste generator device that can recreate taste sensations associated with food, speakers or other auditory devices, and haptic feedback or other touch sensory devices. For example, device 100 may encompass a device used for augmented, virtual, or mixed reality applications, such as a headset, that may be wearable by a user 102. The source 108 may encompass an application server, network server, or other device capable of producing sensory datastreams for processing by the user device 102. For example in a third-generation partnership project (3GPP) network, this source 108 could be a camera, a speaker/headphone, or another party providing data via an evolved Node-B (eNB)/5G Node B (gNB). The network 106 may be a 3GPP-type cellular network, the Internet, or other type of network. In some embodiments, a sensor 104 may be in electronic communication with the user device 102 directly and/or via network 106. In some embodiments, sensor 104 may be in electronic communication with other devices, such as source 108, via network 106. The sensor 104 may have capabilities of, for example, measuring one or more of: HRV, SKT, RRA, FE, BP, GA, EOG, EEG, ECG, GSR, or EMG for user 102 as discussed above.

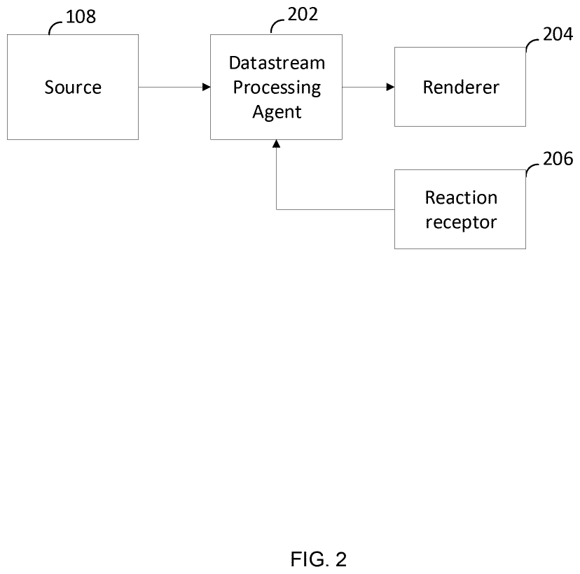

FIG. 2 is a block diagram of components of a system, according to some embodiments. The system may encompass a datastream processing agent 202, a renderer 204, and reaction receptor 206, and source 108 described above in connection with FIG. 1. In some embodiments, datastream processing agent 202 resides in device 100. According to some embodiments, the system learns to enhance or subdue sense-related data contained in datastreams based on the reactions of the user.

Raw datastreams that contain unprocessed levels of sense intensity may be provided by a source 108. For example, in a third-generation partnership project (3GPP) network, this source could be a camera, a speaker/headphone, or another party providing data via an eNB/gNB.

The data processing agent 202 may include a set of components used to learn, based on a user's emotional state and personal preferences, how to adjust the intensity of different senses and modify the raw datastreams from source 108 accordingly. In some embodiments, the set of components are logical. They may be in the user's device 100 or can be hosted by a third-party service that is reachable by a user device 100. In some embodiments, data processing agent 202 may utilize machine learning techniques as described herein, and may include, for example, a neural network.

Processed datastreams may be sent from data processing agent 202 to renderer 204, e.g., to control a sensor actuator in accordance with the processed datastreams. For example, renderer 204 may be VR goggles, glasses, display device, a phone, or other device.

Depending on the technique used to gauge users' reactions, different sensors 104 can also be used and even placed on user's body. The reaction receptor 206 may measure a user's emotional state and/or measure environmental qualities and provide such information to datastream processing agent 202. In some embodiments, reaction receptor 206 may aggregate information from one or more sensors 104.

According to some embodiments, the problem may be formulated as a reinforcement learning problem (i.e., as a Markov Decision Process—MDP with unknown transition probabilities). For reasons of simplicity, a finite MDP is used in the definition, where state and action spaces are finite (see below), however continuous state and action spaces may also be used.

### Action Space

An action space may define the possible set of actions to be taken on the raw datastream. These actions may be discrete, and they indicate the level of intensity over, or below, a reference intensity that a datastream should be adjusted. For example, considering an audio datastream, the possible action space for that audio datastream could be:

$$\\ \begin{matrix}\begin{matrix}[ - 1 , & - 0.8 , & - 0.6 , & - 0.4 , & - 0.2 , & 0 , & 0.2 , & 0.4 , & 0.6 , & 0.8 , & 1 ]\end{matrix} & ( set 1 )\end{matrix}$$

Accordingly, the audio stream level adjustment can be set from completely muted (−1) to double level than it currently is (1). The raw data stream can also remain unchanged, i.e. at 0 value. When it comes to audio datastreams, the level may mean the amplitude of the audio wave, which could be increased or decreased by a percentage indicated by the action. In another embodiment, the level may indicate the pitch of the audio wave (i.e., the frequency)—a higher pitch would indicate that the period of the frequency is reduced, as per the action space above. In yet another embodiment, it is possible that both pitch and amplitude are adjusted, in this case the action space would be double the size of the one indicated in set 1 above, one action for each frequency, period, and amplitude of the sound wave. Depending on the use case, there can be more granular sets than set 1 with smaller step between adjacent values, or even continuous sets of values.

Following the same example of set 1, adjustments can also be made to images, for example video frames of VR/AR world. Hue, brightness and saturation are all parameters that can modified to adjust colors in the image (e.g., to a lighter color scheme) and can all be considered as level of intensity for visual data streams.

When it comes to taste, an indication of intensity of bitter, sour, sweet, salty and umami can be used, as these five are considered basic tastes from which all other tastes are generated. Again, the indications can use set 1 or other with finer/coarser granularity, which, in the case of taste may include 5-ford the size (based on the current commonly agreed five basic tastes).

For odors and smell, there may be no consensus yet as to what primary odors exist. One categorization considers the following odors, which at various levels of intensity may synthesize any smell: Musky, Putrid, Pungent, Camphoraceous, Ethereal, Floral, Pepperminty (Reference [3]). Accordingly, there may be seven sets similar to set 1, each characterizing the intensity of each basic constituent of taste.

Finally, for touch, according to Reference [4], there are four basic senses: pressure, hot, cold and pain. Other sensations are created by a combination of the other four. For instance: the experience of a tickle is caused by the stimulation of neighboring pressure receptors. The experience of heat is caused by the stimulation of hot and cold receptors. The experience of itching is caused by repeated stimulation of pain receptors. The experience of wetness is caused by repeated stimulation of cold and pressure receptors. Again, as in previous cases, a similar set such as set 1 can be used for each of four basic constituents.

### State Space

The state space contains a description as to the current emotional state of the person. For characterizing the state space, the broadness of emotional states that can be detected by one or an ensemble of techniques referenced above may be delineated. According to psychologist Paul Eckman, there exist six types of basic emotions: happiness, sadness, fear, disgust, anger and surprise. This categorization may be used as an example to illustrate how the state description can be serialized and presented to the agent:

$$\\ \begin{matrix}\begin{matrix}[ happiness , & sadness , & fear , & disgust , & anger , & surprise ] =\end{matrix} \begin{matrix}[ 0.6 , & 0.02 , & 0.04 , & 0.02 , & 0.1 , & 0.22 ]\end{matrix} & ( set 2 )\end{matrix}$$

Set 2 illustrates an example state report, which may be the average of an ensemble of techniques referenced above. Applications in controlled environments might be able to choose more accurate but invasive techniques, whereas applications in public environments might want to use non-invasive techniques.

If automated identification of the basic emotions using sensors is impractical (e.g. visual sensors for facial expressions, body posture and gestures are unavailable), a multi-dimensional analysis of emotional states could be used instead. Multi-dimensional analysis pertains to mapping emotions to a limited set of measurable dimensions, for instance valence and arousal. Valence refers to how positive/pleasant or negative/unpleasant a given experience feels, and arousal refers to how energized/activated the experience feels.

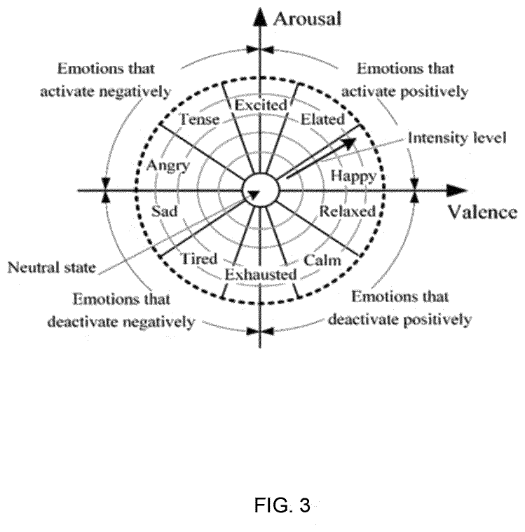

FIG. 3. Illustrates a mapping of emotions to a set of measurable dimensions, according to some embodiments. An example state description using such dimensions could then be serialized as follows:

$$[ valence , arousal ] = [ - 0.4 , + 0.7 ] \qquad ( set 3 )$$

An overview of approaches to emotion recognition and evaluation using techniques such as GSR, HRV, SKT, ECG, and EEG is described in Reference [2]. More or less invasive sensors can be used to measure emotional states along the dimensions: e.g. if the arousal level is increased, the conductance of the skin also increases, the heart rate increases etc.; the latter can be measured using various wearable sensors. What is more, such dimensions and thereby emotional states can be captured using even typical devices such as smartphones via direct user input, for example, using the Mood Meter App.

In addition to leading to more accurate determination of emotional states, multi-dimensional analysis of emotions also potentially reduces the dimensionality of the state space, thus making processing of emotional states computationally cheaper.

The final constituent of the state space, in addition to the emotional states of the user, are environmental qualities that affect the calculation of the reward function. These qualities can be for example the level of ambient noise and lighting, the current temperature, wearables configuration such as the sound of the headset and the brightness of the screen, etc.

### Reward Function Using a Desired State ML Model (DSML)

For RL models, one design decision is to choose a reward function that directs RL agents towards preferred target states by rewarding those states more. When it comes to assessing what is the user target emotional state for a given current emotional state and environmental state is hard in advance. For example, different people have different emotional reaction for the same environment state. Furthermore, sometimes people want to be more exited, while sometimes they want to feel more relaxed depending on environmental and current emotional state.

According to some embodiments, a supporting ML model may be trained to try to learn a user's desired next emotional state for a given current emotional and environmental state. The environmental state can be observed by the user's wearable devices (e.g., headset) or using other devices such a mobile phone. The aforementioned ML model may be called Desired State ML model (DSML).

According to some embodiments, the input vector to the DSML model is a concatenation of user current emotional state vector and a vector of environment state. For instance, if the user emotional state is sᵤₛₑᵣ=[valence=−0.4, arousal=0.7] and the environment state is sₑₙᵥ=[noise=65 dB, light=1000 lux, temp=21C, headset_sound_volume=20%, screen_brightness=70%], then the input vector to DSML is s_{current}=[noise=65 dB, light=1000 lux, temp=21C, headset_sound_volume=20%, screen_brightness=70%, valence=−0.4, arousal=0.7].

The output of DSML is an emotional state desired by the user represented as a vector of its components. The training data for the DSML is collected from the actions taken by user to adjust parameters of an AR/VR/MR experience during use. When users make adjustments, then parameters of the equipment and possibly content is recorded as vector of current environment state and the characteristics of emotional state are measured before and after the adjustment took place. The measured emotional states are recorded as current and desired emotional state. So, each action taken by user creates one input output tuple for the DSML training.

Note that there could be fluctuations in the emotional state measurements so when we talk about emotional state before and after an action, we are talking about averaged values measured for a period of time before and after the action.

### Using DSML to Assess Reward Function of RL Model

The reward for an action taken by RL agent could be computed using results of DSML model in the following way. For any given current state s_{current} RL agent need to suggest an action. The reward each action gets depends on how well the resulting next state sₙₑₓₜ is close to desired state s_{desired} predicted by DSML for s_{current}. Note that s_{current} and sₙₑₓₜ states contain both emotional and environmental components while s_{desired} state has only emotional components. Let's call a corresponding component of s_{curren}, sₙₑₓₜ, s_{desired} as c_{curren}, cₙₑₓₜ, c_{desired}. Then for that component the penalty p_{c} is computed as: p_{c}=|c_{desired}−cₙₑₓₜ|/|c_{curren}−c_{desired}|

Then the total penalty p for the action is the sum over all components of emotional state vector. Finally, the reward for the action is reciprocal of the penalty, i.e. r=1/p.

### Example: Countering Motion Sickness

While the disclosed embodiments target sense perception, the embodiments disclosed herein can also find another application in countering motion sickness that people may experience, for example on passenger cars. For example, a study from the University of Michigan showed a correlation between physiological measurements and motion sickness of car passengers (head position relative to the torso, heart rate, skin temperature etc.).

To counter motion sickness, studies identified that triggering of certain sounds, such as listening to pleasant music (Reference [5]) and correlating engine sounds and vibration with visual flow speed (Reference [6]) could reduce motion sickness. Therefore, the embodiments disclosed herein can be extended with a mechanism to reduce motion sickness.

Specifically, the state space in case of motion sickness mitigation may consist of physiological measurements that indicate the severity of motion sickness. These may include movement of the head (also known as head roll), which is proportional to the severity of the sickness (observed in Reference [5] and Reference [7]). Head roll and pitch can be measured using accelerometer and gyroscope sensors on a wearable such as AR/VR glasses. An increase in tonic and phasic GSR has also been found to contribute to motion sickness in Reference [7]. Another study also identified that changes in the blinking behavior of the eyes and breathing/respiration also suggest an uncomfortable situation that may be linked to motion sickness (Reference [8]).

A serialization of the state space therefore may include one or more of the following, depending also on the type of sensors present:
Degree of change in head posture, i.e., standard deviation or other statistical dispersion measurement of the angle of change of roll axis and pitch axis of the head based on aggregated data points spanning a predefined duration (e.g., 2 min);
Tonic and phasic GSR increase or decrease from previous state; Standard deviation or other statistical dispersion measurement of eye blinks, based on aggregated data points spanning a predetermined duration (e.g., 2 min); and
Standard deviation or other statistical dispersion measurement of respiration events (e.g., “breathe-ins”), based on aggregated data points spanning a predetermined duration (e.g., 2 min).

In terms of the action space, in the simplest most straightforward case, an audio stream will be transmitted playing back some pre-recorded pleasant music (as observed in Reference [5]). In some embodiments, an audio stream will be transmitted playing back music with a tempo correlating to a speed of a vehicle. In another case, a video stream can be created and correlated with the sounds of the engine and vibration of the car, as well as the vehicle's actual speed. The sounds of the engines, vibration and speed can be retrieved by the datastream processing agent 202 from the headset's microphone, accelerometer and GPS receiver respectively and then a video can be synthesized to provide the sense of speed and flow matching those readings. Additional or alternative actions may be taken to counteract detected motion sickness as well, such as, for example, lowering a window, changing a position of a seat, reducing an experience of content (e.g., from three-dimension to two-dimension), adjusting an air conditioner, and/or adjusting an air recycler.

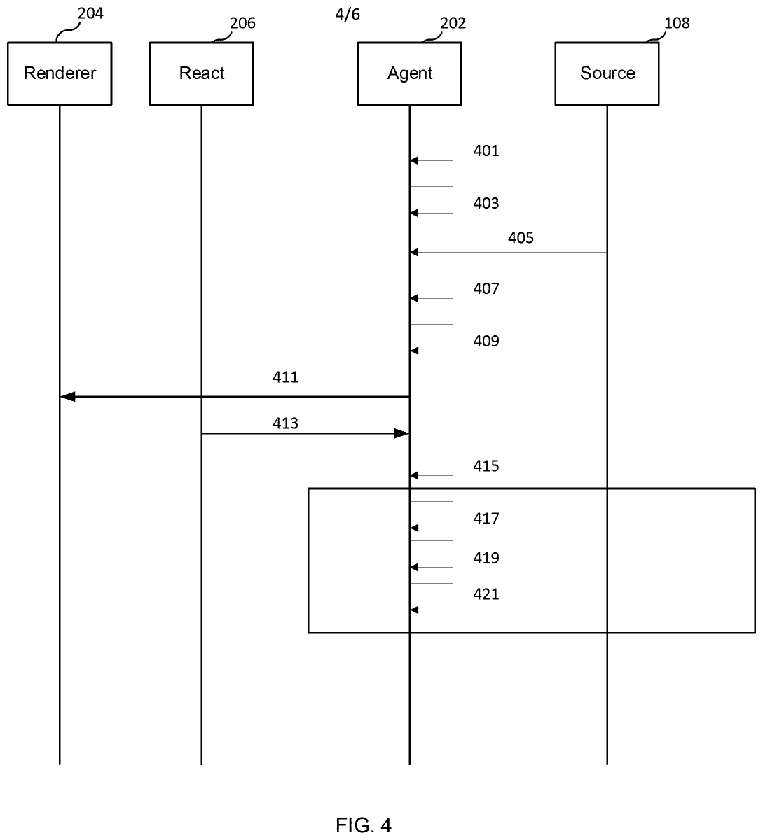

FIG. 4 is a flowchart illustrating a process according to some embodiments. Agent 202 may be rendering agent 202 discussed above, react 206 may be reaction receptor 206 discussed above, renderer 204 may be renderer 204, and source 108 may be source 108 as discussed above in connection with FIG. 2. FIG. 4 illustrates a process of reinforcement learning, according to some embodiments.

At 401, agent 202 initializes a target network tn and a deep q network dqn.

At 403, agent 202 initializes an experience cycle buffer B. Steps 401 and 403 may be used to train a machine learning model as discussed above using observations of an old and new state.

At 405, a raw datastream is received at agent 202 from source 108.

At 407, an action a is selected using a selection policy (e.g., e-greedy). The action may be based on machine learning techniques of exploration and exploitation. In exploration, a random action may be selected. In exploitation, a constrained action may be selected.

At 409, the raw datastream is processed based on the action selected at step 407.

At 411, the processed datastream is provided to renderer 204. The processed datastream may control an actuator of a user device 100 to provide a sensory output to a user.

At 413, the react 206 observes a reward r(i) and new state s(i+1) and provides the observations to agent 202.

At 415, the agent 202 stores <s(i+1), s(i), a, r (i)> in the buffer B.

At predetermined iterations, steps 417, 419, and 421 may be performed by agent 202. In some embodiments, steps 417, 419, and 421 are performed using a convolutional neural network and/or mean-square algorithm in agent 202. At step 417, a random minibatch of experiences <s(j+1), s(j), a, r (j)> are selected from the buffer B.

At step 419, y(j) is set equal to r(j)+ymaxQ(s(j+1), a(j+1), tn).

At step 421, a gradient descent step is performed on tn(y(j)−Q(s(j), a(j), dqn)) ↑2.

Example code for generating the flow in FIG. 4 is reproduced below.

@startuml
title RL-based sense datastream adaptation
participant render
participant react
participant agent
participant source
note over render, source:render: rendering agent\nreact: user emotional state and
environmental qualities receptor\nagent: datastream processing agent\nsource: AR/VR
Environment Content Provider
group Training
agent−>agent: init target network tn,\ndeep q network dqn
agent−>agent: init experience cyclic buffer B
loop for Episode i in K
source−>agent: Raw datastream
agent−>agent: Select action a using selection policy (e.g., e-greedy)
agent−>agent: Process datastream using action a
agent−>render: Processed datastream
react−>agent: observe reward r(i), new state s(i+1)
agent−>agent: Store <s(i+1), s(i), a, r(i)> in B
group every Y iteration
agent−>agent: Select random minibatch of experiences <s(j+1), s(j), a, r(j)> from B
agent−>agent: Set y(j) = r(j)+γmaxQ(s(j+1),a(j+1),tn)
agent−>agent: Perform gradient descent step on tn (y(j)−Q(s(j),a(j),dqn))↑2
end
group every M iteration, M >> Y
agent−>agent: copy tn weights to dqn
end
end
end
group Execution
source−>agent: Raw Datastream
react−>agent: Status
agent−>agent: Select action a based on status by DQN execution
agent−>agent: Process datastream using action
agent−>render: Processed datastream
end
@enduml

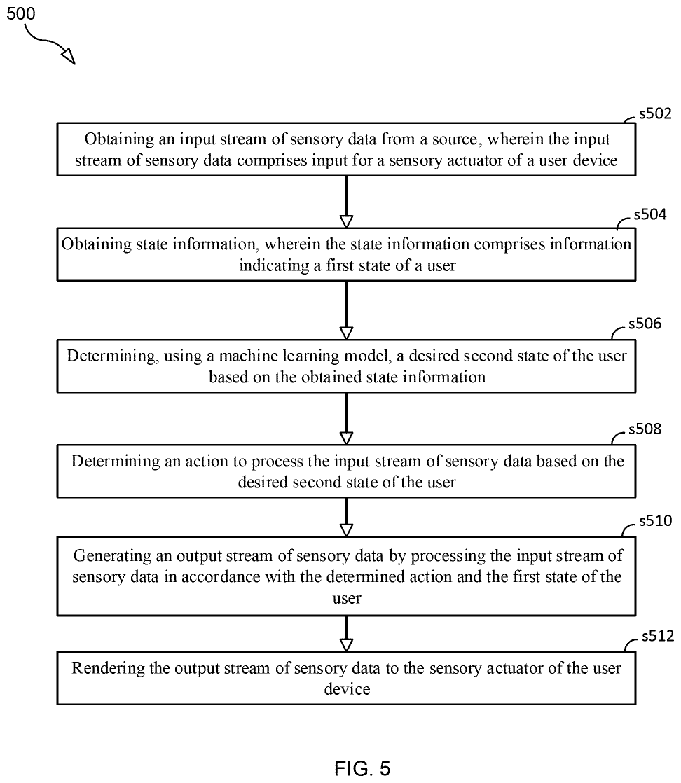

FIG. 5 is a flowchart illustrating a method according to some embodiments. According to some embodiments, method 500 is a computer-implemented method of processing a stream of sensory data. The method 500 includes step s502 of obtaining an input stream of sensory data from a source, wherein the input stream of sensory data comprises input for a sensory actuator of a user device. The method 500 includes step s504 of obtaining state information, wherein the state information comprises information indicating a first state of a user. The method 500 includes step s506 of determining, using a machine learning model, a desired second state of the user based on the obtained state information. In some embodiments, the determining may encompass predicting the desired second state of the user based on the obtained state information. The method 500 includes step s508 of determining an action to process the input stream of sensory data based on the desired second state of the user. The method 500 includes the step s510 of generating an output stream of sensory data by processing the input stream of sensory data in accordance with the determined action and the first state of the user. The method 500 includes step s512 of rendering the output stream of sensory data to the sensory actuator of the user device.

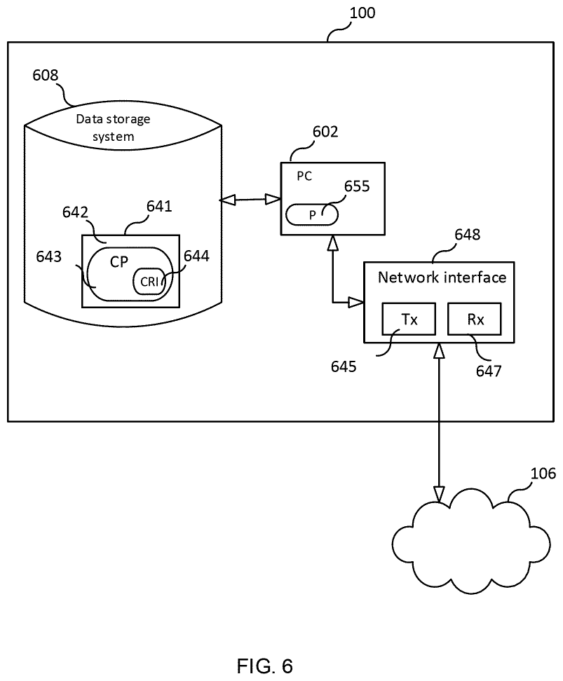

FIG. 6 is a block diagram of an apparatus 100 according to an embodiment. In some embodiments, apparatus 100 may be user device 100 described above in connection with FIGS. 1-2. As shown in FIG. 6, apparatus 100 may comprise: processing circuitry (PC) 602, which may include one or more processors (P) 655 (e.g., one or more general purpose microprocessors and/or one or more other processors, such as an application specific integrated circuit (ASIC), field-programmable gate arrays (FPGAs), and the like); communication circuitry 648, comprising a transmitter (Tx) 645 and a receiver (Rx) 647 for enabling apparatus 100 to transmit data and receive data (e.g., wirelessly transmit/receive data); and a local storage unit (a.k.a., “data storage system”) 608, which may include one or more non-volatile storage devices and/or one or more volatile storage devices. In embodiments where PC 602 includes a programmable processor, a computer program product (CPP) 641 may be provided. CPP 641 includes a computer readable medium (CRM) 642 storing a computer program (CP) 643 comprising computer readable instructions (CRI) 644. CRM 642 may be a non-transitory computer readable medium, such as, magnetic media (e.g., a hard disk), optical media, memory devices (e.g., random access memory, flash memory), and the like. In some embodiments, the CRI 644 of computer program 643 is configured such that when executed by PC 602, the CRI causes device 100 to perform steps described herein (e.g., steps described herein with reference to the flow charts). In other embodiments, device 100 may be configured to perform steps described herein without the need for code. That is, for example, PC 602 may consist merely of one or more ASICs. Hence, the features of the embodiments described herein may be implemented in hardware and/or software.

While various embodiments are described herein, it should be understood that they have been presented by way of example only, and not limitation. Thus, the breadth and scope of this disclosure should not be limited by any of the above described exemplary embodiments. Moreover, any combination of the above-described elements in all possible variations thereof is encompassed by the disclosure unless otherwise indicated herein or otherwise clearly contradicted by context.

Additionally, while the processes described above and illustrated in the drawings are shown as a sequence of steps, this was done solely for the sake of illustration. Accordingly, it is contemplated that some steps may be added, some steps may be omitted, the order of the steps may be re-arranged, and some steps may be performed in parallel.

### ABBREVIATIONS

RL Reinforcement Learning
AR/VR Augmented Reality/Virtual Reality
EEG Electroencephalography
ECG Electrocardiogra
GSR Galvanic Skin Response
HRV Heart Rate Variability
RRA Respiration Rate Analysis
SKT Skin temperature measurements
EMG Electromyogram
EOG Electrooculography
FE Facial Expressions

### REFERENCES

[1] https://www.ericsson.com/4ae13b/assets/local/reports-papers/consumerlab/reports/2019/10hctreport2030.pdf
[2] Dzedzickis A, Kaklauskas A, Bucinskas V. Human Emotion Recognition: Review of Sensors and Methods. Sensors (Basel). 2020; 20 (3): 592. Published Jan. 21, 2020. doi: 10.3390/s20030592, https://www.ncbi.nlm.nih.gov/pmc/articles/PMC7037130/
[3] Auffarth, B. (2013). [Review of Understanding smell: the olfactory stimulus problem]. Neuroscience and Biobehavioral Reviews, 37 (8), 1667-1679. https://doi.org/10.1016/j.neubiorev.2013.06.009
[4] https://opentextbc.ca/introductiontopsychology/chapter/4-4-tasting-smelling-and-touching/
[5] Less sickness with more motion and/or mental distraction, Jelte E. Bos, Journal of Vestibular Research 25 (2015) 23-33 23 DOI 10.3233/VES-150541 IOS Press
[6] Sawada, Y., Itaguchi, Y., Hayashi, M. et al. Effects of synchronised engine sound and vibration presentation on visually induced motion sickness. Sci Rep 10, 7553 (2020). https://doi.org/10.1038/s41598-020-64302-y
[7] Irmak, T., Pool, D. M. & Happee, R. Objective and subjective responses to motion sickness: the group and the individual. Exp Brain Res 239, 515-531 (2021). https://doi.org/10.1007/s00221-020-05986-6
[8] Mark S. Dennison, A. Zachary Wisti, Michael D'Zmura, Use of physiological signals to predict cybersickness, Displays, Volume 44, 2016, Pages 42-52, ISSN 0141-9382, https://doi.org/10.1016/j.displa.2016.07.002.
[9] U.S. Pat. No. 9,355,356B2-Apparatus and methods for capturing and generating user experiences
[10] U.S. Pat. No. 8,243,099B2-Method and system for haptic interaction in augmented reality

1. A computer-implemented method of processing a stream of sensory data, the method comprising:
obtaining an input stream of sensory data from a source, wherein the input stream of sensory data comprises input for a sensory actuator of a user device;
obtaining state information, wherein the state information comprises information indicating a first state of a user;
determining, using a machine learning model, a desired second state of the user based on the obtained state information;
determining an action to process the input stream of sensory data based on the desired second state of the user;
generating an output stream of sensory data by processing the input stream of sensory data in accordance with the determined action and the first state of the user; and
rendering the output stream of sensory data to the sensory actuator of the user device.

2. The method of claim 1, wherein the state information further comprises information indicating a state of an environment of the user.

3. The method of claim 2, wherein the state of an environment of the user comprises one or more of: a level of ambient noise, a level of lighting, a current temperature, a sound of an engine, a vibration of a vehicle, a speed of a vehicle, a configuration of one or more wearable devices, a height above sea level, barometric pressure, humidity, or gas concentration.

4. The method of claim 1, wherein the source comprises one or more of: a camera, a speaker, a headphone, or a network node.

5. The method of claim 1, wherein the state information is obtained from one or more sensors.

6. The method of claim 1, wherein the determined action is one of a plurality of discrete actions from a predefined action space.

7. The method of claim 6, wherein the plurality of discrete actions comprises an adjustment to a level of the input stream of sensory data and the predefined action space comprises a range of possible levels of the input stream of sensory data.

8. The method of claim 7, wherein the input stream of sensory data comprises one or more of a stream of audio data, a stream of visual data, a stream of gustatory data, a stream of olfactory data, or a stream of tactile data, and
wherein the input stream of sensory data is broken into one or more constituents.

9. The method of claim 8, wherein the one or more constituents comprise one or more of:
amplitude, frequency, pitch, timbre, or duration for the stream of audio data,
hue, brightness, lightness, or saturation for the stream of visual data,
bitter, sour, sweet, salty, or umami for the stream of gustatory data,
musky, putrid, pungent, camphoraceous, ethereal, floral, pepperminty, fragrant, woody/resinous, fruity (non-citrus), chemical, sweet, popcorn, lemon, or decayed for the stream of olfactory data, or
pressure, heat, chill, or pain for the stream of tactile data.

10. The method of claim 1, wherein the first state of a user and the desired second state of the user correspond to one or more of:
happiness, sadness, fear, disgust, anger, or surprise, or
a measure of valence and a measure of arousal.

11. The method of claim 1, wherein the machine learning model comprises an unsupervised reinforcement learning.

12. The method of claim 11, further comprising training the reinforcement learning model using a plurality of training samples, wherein each training sample comprises:
an action used to process an input stream of sensory data,
a state of the user before the action, and
a state of the user after the action.

13. The method of claim 1, further comprising:
obtaining second state information, wherein the second state information comprises information indicating a third state of a user.

14. The method of claim 13, further comprising calculating a penalty (p) or reward (r) for the machine learning model.

15. The method of claim 14, wherein the calculating the penalty (p) or reward (r) comprises the following formula:
$$\\ p = \vert {S}_{desired} - {S}_{next} \vert / \vert {S}_{current} - {S}_{desired} \vert ,$$
$$\\ r = 1 / p ,$$
S_{desired} is the determined desired second state of the user,
Sₙₑₓₜ is the third state of the user, and
S_{current} is the first state of the user.

16. The method of claim 1, wherein
the information indicating the first state of the user comprises one or more physiological measurements indicative of motion sickness, and
the determined desired second state of the user comprises one or more physiological measurements not indicative of motion sickness.

17. The method of claim 16, wherein the input stream of sensory data is a stream of audio data, and the determined action comprises one of:
processing the stream of audio data to play predetermined music or
processing the stream of audio data to correlate a tempo of music with a speed of a vehicle.

18. The method of claim 16, wherein the input stream of sensory data is a stream of visual data, the state information further comprises information indicating a speed or flow of a vehicle, and the determined action comprises correlating the stream of visual data with the speed or flow of the vehicle.

19. The method of claim 16, further comprising one or more of:
lowering a window;
changing a position of a seat;
reducing an experience of content from three-dimension to two-dimension;
adjusting an air conditioner; or
adjusting an air recycler.

20. A device adapted to perform the method according to claim 1.

21-22. (canceled)
